(19) United States
(12) Patent Application Publication
(10) Pub. No.: US 2014/0275836 A1
(43) Pub. Date: Sep. 18, 2014

(54) DEVICE TO MONITOR AND PROMOTE SUCCESSFUL ENDOTRACHEAL INTUBATION AND VENTILATION

(71) Applicant: Los Angeles Biomedical Research Institute at Harbor-UCLA Medical Center, Torrance, CA (US)

(72) Inventor: Ruey-Kang Chang, Diamond Bar, CA (US)

(73) Assignee: Los Angeles Biomedical Research Institute at Harbor-UCLA Medical Center, Torrance, CA (US)

(21) Appl. No.: 14/206,137

(22) Filed: Mar. 12, 2014

Related U.S. Application Data
(60) Provisional application No. 61/788,616, filed on Mar. 15, 2013.

(51) Int. Cl. A61M 16/04; A61B 5/00; A61B 7/04; A61B 5/113; A61B 7/00

(52) U.S. Cl. CPC A61M 16/0488; A61B 5/113; A61B 7/003; A61B 7/04; A61B 5/0004; A61B 5/4836
USPC 600/301

(57) ABSTRACT

An apparatus including an accelerometer and a microphone contained in a housing that can be placed on a chest of an individual, the accelerometer configured to detect chest motion and microphone to detect respiratory sounds during at least one of inspiration and expiration. A method including placing such an apparatus on a chest of a subject. A method of monitoring an intubation including simultaneously assessing chest movement and air movement sounds in the airway and lungs; and indicating the status of the intubation based on the assessing. A machine-readable medium containing non-transitory program instructions that, when executed, cause a processor to perform a method including assessing chest movement and air movement sounds in a patient; and indicating the status of the intubation based on the assessing.

### CROSS-REFERENCE TO RELATED APPLICATION

The application claims the benefit of the earlier filing date of co-pending U.S. Provisional Patent Application No. 61/788,616, filed Mar. 15, 2013 and incorporated herein by reference.

### BACKGROUND

Patient safety is one of the most important challenges facing today's healthcare environment, and is a top priority for improvement of the quality of care. Inadvertent esophageal intubation and unsuccessful endotracheal intubation that is not recognized in time continue to cost thousands of lives each year. These events occur regularly in the intensive care unit (ICU), operating room, emergency department, and in many circumstances, at the scene of emergency events by first responders.

Current standards for clinical practice to confirm a successful endotracheal intubation rely on the clinician to evaluate adequate chest rise with each inspiration and airway flow sounds on auscultation of the lungs. However, current practice does not prevent many of the possible human errors that can jeopardize patient safety. The apparent problems with current practice are: 1) a clinician neglects to check on chest rise and breath sounds; 2) a wrong assessment or inadequate experience of the clinician; 3) the time involved in the process may delay resuscitation effort; and 4) the assessment of an endotracheal intubation determination is not always possible in a crowded noisy (frantic) situation.

The use of capnography devices has gained wide popularity by many clinicians. Capnography devices are designed to detect CO₂ coming out of the endotracheal tube during expiration. Problems with use of such devices include: 1) the additional time and extra steps need for such confirmation; and 2) the possibility of non-detection of expiratory CO₂ in a patient in full cardiac arrest. Furthermore, capnography devices cannot detect conditions such as right main stem intubation and pneumothorax.

### BRIEF DESCRIPTION OF THE DRAWINGS

FIG. 1 is a cross-sectional top view of an embodiment of a device for assessing intubation.

FIG. 2 is a cross-sectional side view of the device of FIG. 1.

FIG. 3 is a top, side sectional view of the device of FIG. 1.

FIG. 4A shows an embodiment of the device of FIG. 1 on a subject's chest during inspiration.

FIG. 4B shows an embodiment of the device of FIG. 1 on a subject's chest during expiration.

FIG. 5 shows a flow chart of an embodiment of a method of monitoring and assessing intubation.

### DETAILED DESCRIPTION

A device for use in assessing intubation is disclosed, as is a method for assessing intubation. In one embodiment, a device includes an accelerometer and one or more microphones contained in a housing that can be placed on a subject's chest. The device assesses intubation by 1) chest rise; and 2) airway sounds by auscultation. In one embodiment of the device, the chest rise will be assessed by a tri-axial accelerometer, and airway sounds will be assessed by an acoustic microphone.

Chest rise can be difficult to assess by inexperienced clinicians, in a crowded, busy and distractive setting or environment with inadequate lighting. However, chest rise can be precisely and objectively assessed by an accelerometer. A typical tri-axial accelerometer can detect acceleration and deceleration from all 3 axes at a sensitivity of less than one millimeter (mm).

FIG. 1 and FIG. 2 show an embodiment of a device. FIG. 1 is a top cross-sectional view and FIG. 2 is a side cross-sectional view. In this embodiment, device 100 is housed in round, disk shaped enclosure 110 of, for example, a metal material such as aluminum or a hard plastic material having representative dimensions on the order of 40 mm diameter, d, and 15 mm height, h, defined by a sidewall or sidewalls. Connected to a surface of enclosure 110, surface of intended skin contact, is diaphragm 120. Diaphragm 120 includes, for example, a diaphragm member of, for example, a hard epoxy with an over-molded silicon flexible surround that may be formed fitted or adhesively connected to sidewalls of enclosure 110. In one embodiment, diaphragm 120 is similar to acoustic diaphragms used in conventional stethoscopes, and is intended to augment the acoustic signals from respiration. The skin contact surface can be adhered to the skin using a vacuum suction mechanism, or by a disposable cover with adhesive hydrogel.

Disposed within enclosure 110 of device 100 in this embodiment are accelerometer 130, microphone sensor 140, microphone sensor 150, microcontroller unit 140 and button 170. In one embodiment, accelerometer 130 is a tri-axial accelerometer based on the HDK HAAM-372. These accelerometers have the range of ±2 g and ±8 g, and a digital output that minimizes noise. The accelerometers feature programmable threshold detection, such that a microcontroller unit (MCU) can be put to sleep and awakened by motion triggering. In one embodiment, an accelerometer-only, gyroscope-free inertial measurement unit (GF-IMU) will be used for motion tracking (see EcoIMU: A Dual Triaxial-Accelerometer Inertial Measurement Unit for Wearable Application by Yi-Lung Tsai, et al., Proc. International Conference on Body Sensor Networks (BSN 2010), Singapore (Jun. 7-9, 2010), pp. 207-212). Accelerometer 130 is configured to detect motion, specifically, chest motion during at least one of inspiration and expiration. An algorithm for motion detection based on accelerometer data has been developed and tested.

In one embodiment, microphone sensor 140 and microphone sensor 150 are electret or piezo sensors. Microelectromechanical (MEM) sensors can also be used. In one embodiment, microphone sensor 140 is placed in the center of enclosure 110 corresponding to (e.g., sensor facing) the skin contact surface of the device with acoustic diaphragm 120 to augment the respiratory sound. Microphone sensor 150 may be placed at a top surface of the device facing the ambient environment, in order to detect ambient noise. In one embodiment, ambient noise detected by microphone sensor 150 is used for noise subtraction (e.g., subtracted from sound detected by microphone sensor 140) to enhance the respiratory sound signals. In another embodiment, the two microphone sensors of device 100 may be replaced with a single microphone to detect respiratory sounds. In one embodiment including a single microphone sensor, the microphone sensor includes noise subtraction functionality to reduce the presence of non-respiratory sounds (e.g., ambient sounds).

In addition to the sensors (accelerometer and one or more microphones), device 100 also includes microcontroller unit (MCU) 160 for signal processing based on a predetermined algorithm. MCU 160 is communicatively connected to the sensors of device 100. Battery 170 of, for example, a lithium polymer type is also included and is connected to MCU 160 and the sensors to provide power to the device. Lithium polymer batteries have high charge density and good power density, which are needed for burst (peak-power) processing patterns. Such a battery can be made as lightweight as 1.2 grams with a capacity of 90 mAh at 3.7-4.2 V. In one embodiment, battery strength is monitored periodically by MCU 160. Battery 170 can be recharged using conductive (line in) or inductive (wireless) mechanisms.

A representative algorithm includes a set of non-transitory instructions to query and/or receive signals from the sensors and to transmit signals. MCU 160 may also include a memory (e.g., flash memory such as micro-SD card) to record receipt and transmission of sensor signals. In one embodiment, wireless transmitter, based on radiofrequency such as Bluetooth 4.0 protocol, is installed for transmission of chest rise and sound signals to a remote receiver. The transmitted data on monitoring of the endotracheal intubation procedure may be integrated with the patient's electronic medical record (EMR) for recording keeping and documentation, and for offline review as a part of quality assurance and education.

On and optionally protruding from a surface of enclosure 110 opposite diaphragm 120 are power button 175, indicator light 180, and dial 185, as shown in FIG. 3. Each of power button 175, indicator 180 and dial 185 are connected to MCU 160. Power button 175 is used to turn device 100 on and, in one embodiment, is connected to battery 170. Once the power is on, in one embodiment, indicator light 180 will flash red once per second. Indicator light 180 changes to amber or green color based on the sensor detection signals transmitted to indicator light 180 by MCU 160 (discussed in Table 1). Because the size of patients varies, the sensitivity of chest rise as well as airway sounds can be adjusted in order to optimize the algorithm for detection. In one embodiment, device 100 includes selector 185 such as a dial switch on the top surface to select among Infant, Pediatric, and Adult patient. Selector 185 is electronically connected to MCU 160. Selection of a sensitivity transmits a signal to MCU 160. Alternatively, non-transitory instructions in MCU 160 direct MCU to query selector 185 for a position of the dial switch. In one embodiment, the machine-readable instructions in MCU 160 include sensitivity instructions dependent on the status of a subject. Such sensitivity instructions are used to trigger a signal to indicator light 180. For an adult patient or subject, a threshold to change indicator light 180 from an amber to a green color based on sensor signals will be greater than for a pediatric or infant patient or subject. A threshold for a pediatric patient or subject will likewise be greater than for an infant. MCU 160 is configured to respond to a sensitivity selection.

Device 100 can be used by emergency first responders, paramedics, emergency room physicians, nurses, respiratory therapist, intensivists, anesthesiologists, and clinicians in any of the settings that endotracheal intubation takes place. Device 100 can be bundled with a laryngoscope in an intubation tray; or as a standalone device to be kept in stock in a crash cart, in the operating room, or in the ambulance. In addition, this can also be a pocket device a clinician who is frequently involved in intubation procedures (e.g., anesthesiologists, respiratory therapists) carries on a daily basis.

Device 100 is to be placed on the patient's left chest, to the left of sternum at 4^{th} intercostal space (medial to the left nipple). The unconscious patient should be supine, with chest exposed, and without movement interference (chest compression). In one embodiment, the user presses power button 175 first, then firmly places device 100 on the left chest and attaches the device using suction or disposable adhesive surface 120. The user proceeds with intubation by placing an endotracheal tube while the indicator light flashes red once per second. The sensors of device 100 continue to monitor the charges (presence and absence of chest rise and airway sounds). In one embodiment, once the user thinks the endotracheal tube is in place, a self-inflatable bag-valve device is connected to the endotracheal tube for ventilation. Once the sensors detect definitive signals indicating three consecutive respiratory cycles of chest rise and inspiration/expiration, the indicator light will change to a solid green.

FIG. 4A shows an embodiment of a device for use in assessing intubation on a subject's chest during inspiration. Device 100, in one embodiment, is placed at the fourth intercostal space, medial to the nipple on the left chest. As shown in FIG. 4A, during inspiration, the chest rises. A rise of the chest can be detected by an accelerometer associated with device 100, and an inspiratory sound is detected by a microphone associated with device 100. FIG. 4B shows the subject's chest during expiration. During expiration, the chest falls which is detected by the accelerometer associated with device 100, and an expiratory sound is detected by a microphone associated with device 100.

Table 1 summarizes the algorithms for detection of different clinical conditions, including esophageal intubation, right main stem intubation, and possible pneumothorax.

TABLE 1
Algorithm of detection based on sensor input

| | Acceler- | | | | |
|---|---|---|---|---|---|
| | ometer | Microphone 140 | | | |
| | 130 | bronchial sounds | | | Indicator |
| Condition | Chest rise | I | E | Interpretation | light 180 |
| 1 | + | + | + | Endotracheal | Green |
| | | | | intubation | |
| 2 | −− | distant | distant | Right main stem | Amber |
| | | | | intubation | |
| 3 | −− | distant | −− | Esophageal | Red |
| | | | | intubation | |
| 4 | −− | −− | −− | Esophageal | Red |
| | | | | intubation or | |
| | | | | blocked | |
| | | | | endotracheal tube | |
| 5 | +/− | + | −− | Pneumothorax | Red |
| 6 | + | −− | −− | Movement | Red |
| | | | | artifact | |

I: inspiratory;
E: expiratory.

FIG. 5 is a flow chart of an embodiment of a method of monitoring and assessing endotracheal intubation. The method is stored in the form of non-transitory machine-readable instructions in MCU 160 of device 100 and executed by MCU 160. Referring to FIG. 5, in this embodiment, method 200 is initiated when device 100 is powered on (block 210). Next, MCU 160 determines whether the patient to be monitored is an infant, pediatric or adult patient based on signal(s) sent to or received from selector 185 and, based on the patient status, configures its sensor detection thresholds (block 220). MCU 160 then verifies sensor input at microphone sensor 140, microphone sensor 150 and accelerometer sensor 130 (block 225, block 230). MCU 160 then queries the sensors for chest rise and bronchial breath sounds (block 235). MCU 160 simultaneously assesses the chest movement and air movement sounds (bronchial breath sounds) provided by the sensor data. If both are absent, MCU 160 directs indicator 180 on device 100 to flash red at a rate of one flash per second (block 240). During this time, MCU 160 again queries the sensor until chest rise and bronchial breath sounds are detected. If chest rise and bronchial breath sounds are detected by the sensors, MCU 160 begins an analysis of inspiration and expiration (block 250). MCU 160 queries the sensors for three respiratory cycles of chest movements and corresponding inspiratory/expiratory breath sounds (block 260). If the sensors indicate three respiratory cycles of chest movements and corresponding inspiratory/expiratory breath sounds, MCU 160 directs indicator 180 on device 100 to illuminate a solid green color indicating successful intubation (block 265).

Where the sensors do not indicate three respiratory cycles of chest movements and corresponding inspiratory/expiratory breath sounds, MCU analyzes the sensor signals for conditions other than successful intubation (block 270). In one embodiment, MCU 160 analyzes for conditions of right main stem intubation, esophageal intubation, blocked endotracheal tube, pneumothorax, or chest movement artifacts (e.g., chest movement associated with an extraneous action such as chest compressions) (block 275). If any of the conditions are detected, MCU 160 directs indicator 180 to illuminate an indicator light of red (or, in one embodiment, amber in the case of right main stem intubation) (block 280). MCU 160 will continue monitoring the sensors and directing indicator 180 until device 100 is powered off.

In another embodiment of a device, a display screen such as a liquid crystal display (LCD) or light emitting diode (LED) screen is placed on the top surface of device 100. In addition to the indicator light, display screen 190 can visually display the results of accelerometer and microphone airway sounds, and the interpretation of the sensor inputs listed in Table 1. In one embodiment, MCU 160 can direct screen to display the condition detected by analysis of the sensor data in text form in display screen 190. The interpretations of the results displayed on the display screen may assist clinicians with assessment and decision making in the events of possible right main stem intubation, blocked endotracheal tube or pneumothorax.

In some settings, such as pre-hospital care by paramedics, supraglottic (or extra-glottic) airway (SGA) devices are used. Examples of SGA devices include laryngeal mask airway (LMA), Combi-tube, and King LT airways. In these devices, a tube is placed in the esophagus or in the oropharynx instead of the trachea. However, successful placement of SGA devices (successful intubation) and effective ventilation can be assessed by the same method of combined input using chest movement and air movement sounds. Therefore, the proposed device, method and algorithm will be as effective for detecting SGA placement and ventilation as they do for endotracheal intubation.

Advantages of a device such as described for assessing intubation include that the device requires no skill or training to use (operator independent and fool proof); results are clearly presented by indicator light; saves time repeated assessments of the chest rise and auscultation; detects right main stem intubation, and possibly pneumothorax; standardizing intubation assessment procedure; and easy documentation (a check box on paper or in EMR), the device can be easily integrated with the Quality Improvement protocols in the operating room, emergency department, or ICU to reduce rate of unintended and unrecognized esophageal or right main stem intubation.

In the description above, for the purposes of explanation, numerous specific details have been set forth in order to provide a thorough understanding of the embodiments. It will be apparent however, to one skilled in the art, that one or more other embodiments may be practiced without some of these specific details. The particular embodiments described are not provided to limit the invention but to illustrate it. The scope of the invention is not to be determined by the specific examples provided above but only by the claims below. In other instances, well-known structures, devices, and operations have been shown in block diagram form or without detail in order to avoid obscuring the understanding of the description. Where considered appropriate, reference numerals or terminal portions of reference numerals have been repeated among the figures to indicate corresponding or analogous elements, which may optionally have similar characteristics.

It should also be appreciated that reference throughout this specification to “one embodiment”, “an embodiment”, “one or more embodiments”, or “different embodiments”, for example, means that a particular feature may be included in the practice of the invention. Similarly, it should be appreciated that in the description various features are sometimes grouped together in a single embodiment, figure, or description thereof for the purpose of streamlining the disclosure and aiding in the understanding of various inventive aspects. This method of disclosure, however, is not to be interpreted as reflecting an intention that the invention requires more features than are expressly recited in each claim. Rather, as the following claims reflect, inventive aspects may lie in less than all features of a single disclosed embodiment. Thus, the claims following the Detailed Description are hereby expressly incorporated into this Detailed Description, with each claim standing on its own as a separate embodiment of the invention.

What is claimed is:

1. An apparatus comprising an accelerometer and a microphone contained in a housing that can be placed on a chest of an individual, the accelerometer configured to detect chest motion and the microphone to detect respiratory sounds during at least one of inspiration and expiration.

2. The apparatus of claim 1, further comprising an indicator coupled to the housing and configured to respond to signals produced by at least one of the accelerometer and the microphone.

3. The apparatus of claim 1, further comprising a control unit and an indicator, the control unit coupled to the accelerometer and the microphone and configured to receive signals from the accelerometer and the microphone and to transmit a signal to the indicator.

4. The apparatus of claim 1, further comprising a control unit and a wireless transmitter, the control unit coupled to the accelerometer and the microphone and configured to receive signals from the accelerometer and the microphone and save the data on a flash memory or transmit data wirelessly to a receiver to be integrated with electronic medical record.

5. The apparatus of claim 3, further comprising a selector coupled to the MCU and configured to indicate a sensor sensitivity threshold based on a status of a patient of infant, pediatric or adult.

6. The apparatus of claim 3, further comprising a selector coupled to the MCU and configured to indicate a sensor sensitivity threshold based on a status of a patient of infant, pediatric or adult.

7. A method comprising:
placing a device on a chest of a subject, the device comprising an accelerometer and a microphone contained in a housing, the accelerometer configured to detect chest motion during at least one of inspiration and expiration; and microphone configured to detect air movement sounds during at least one of inspiration and expiration.

8. The method of claim 5, further comprising intubating the subject.

9. A method of monitoring an intubation comprising:
simultaneously assessing chest movement and air movement sounds in the airway and lungs; and
indicating the status of the intubation based on the assessing.

10. The method of claim 9, wherein chest movement is assessed by a first sensor, and air movement sounds are assessed by a second sensor.

11. The method of claim 10, wherein the first sensor comprises a tri-axial accelerometer.

12. The method of claim 10, wherein the second sensor comprises a microphone.

13. The method of claim 9, wherein the intubation is endotracheal intubation.

14. The method of claim 9, wherein the intubation is selected from esophageal intubation and oropharynx intubation.

15. The method of claim 13, further comprising, wherein intubation is not detected, assessing at least one of right main stem intubation, blocked endotracheal tube, esophageal intubation and pneumothorax,

16. A machine-readable medium containing non-transitory program instructions that, when executed, cause a processor to perform a method comprising:
assessing chest movement and air movement sounds in a patient; and
indicating the status of the intubation based on the assessing.

17. The machine-readable medium of claim 16, wherein the method further comprises:
intubation is endotracheal intubation.

18. The machine-readable medium of claim 16, wherein intubation is selected from esophageal intubation and oropharynx intubation.

19. The machine-readable medium of claim 16, wherein intubation is not detected, the method further comprising assessing at least one of right main stem intubation, blocked endotracheal tube, esophageal intubation and pneumothorax.
